(19) United States
(12) Patent Application Publication
(10) Pub. No.: US 2012/0059441 A1
(43) Pub. Date: Mar. 8, 2012

(54) PHOTOTHERAPEUTIC DEVICE AND SYSTEM

(75) Inventors: David D. Chang, Encino, CA (US); Setsuo Tanaka, Torrence, CA (US); Jake Pananen, Santa Monica, CA (US)

(73) Assignee: Lemer Medical Devices, Inc., Los Angeles, CA (US)

(21) Appl. No.: 13/037,033

(22) Filed: Feb. 28, 2011

Related U.S. Application Data
(60) Provisional application No. 61/310,550, filed on Mar. 4, 2010.
(60) Provisional application No. 61/310,560, filed on Mar. 4, 2010.

(51) Int. Cl. A61N 5/06

(52) U.S. Cl. 607/94; 607/90

(57) ABSTRACT

Phototherapeutic systems with an integral, or adapted for use with an, optical calibration system. A dynamic programmable phototherapeutic treatment system, wherein the treatment device is responsive to a stored treatment plan to generate a predetermined therapeutic UV light signal for application to a treatment surface region of a patient/user.

### REFERENCE TO RELATED APPLICATIONS

This application is related to U.S. Provisional Patent Application Ser. No. 61/310,550, entitled PHOTOTHERAPEUTIC DEVICE WITH OFF-AXIS INTEGRATING LIGHT METER, filed on Mar. 4, 2010 (Attorney Docket No LNLL-0116PR), and U.S. Provisional Patent Application Ser. No. 61/310,560, entitled PHOTOTHERAPEUTIC SYSTM WITH A PATIENT-AND PHYSICIAN-CONTROLLED DYNAMIC TREATMENT PLAN, filed on Mar. 4, 2010 (Attorney Docket No LNLL-0117PR). Both Provisional patent applications are incorporated fully herein by reference.

### FIELD

The invention relates generally to the field of phototherapeutic devices and systems, and more specifically to how therapeutic doses are delivered, adjusted dynamically based on patient feedback, and the light meters designed for use with such devices and systems for enabling control of the same.

### BACKGROUND

Inflammatory diseases of the skin affect a significant portion of the population resulting in significant morbidity. Psoriasis, for example, affects at least 2% of the population. Past and current methods of treatment of skin psoriasis include the application of tars, salicylic acid, steroids, ultraviolet light (phototherapy), and a combination of ultraviolet light used in conjunction with photoactive compounds (photochemotherapy).

Phototherapy involves UV irradiation of the affected skin area, including the scalp. For example psoriasis has been treated with ultraviolet-B (UV-B) light having wavelengths from 290-320 nm. Other skin diseases which have been treated successfully with ultraviolet light include eczema mycosis fungoides, and lichen planus. In addition, ultraviolet light may have a role in the treatment of seborrheic dermatitis.

Lerner et al. (U.S. Pat. No. 5,300,097) describes a phototherapy device that delivers ultraviolet (UV) light to a target area. That device includes a UV light source, which delivers the light to the target area using an array of optical fibers. Using the device typically requires an appointment with a doctor or other trained specialist familiar with the appropriate treatment protocol for the given condition. Because of the inconvenience, cost, and unreliability of restricting use of a phototherapy device to a trained specialist, home use phototherapy devices are being developed.

The amount of UV light that a patient can tolerate depends on their skin type. Individuals with lighter skin can tolerate lower levels of UV light exposures, because they tend to burn relatively easily. Individuals with darker skin can tolerate higher levels of UV light exposure. The amount of UV light used during phototherapy or photochemotherapy must be controlled for, among other variables, different skin types of the end user. In order to assess and control the levels of light emitted by a phototherapeutic device, particularly on design for home use, it is important that the device output be calibrated at least initially, before use, and generally from time to time. Typically in the prior art, an UV light meter is used to calibrate the output of a UV phototherapy device, specifically, the output of UV light used during phototherapy.

In typical phototherapy involving delivery of ultraviolet light, the patient's skin absorbs the UV light becomes relatively tanned and develops hyperplasia (skin thickening). As the skin becomes acclimatized to UV light, a higher level of UV light then is needed to deliver the same amount of effective, therapeutic dosage. The amount of UV light increase or decrease depends on the erythema effect from the previous treatment and pain tolerance of the individual patient. Too much UV light exposure may cause skin redness, irritation, blistering, or severe sun burn. Too little UV light exposure offers little or no therapeutic value. However, making such an adjustment cannot be done easily by the patient, because adjustment of the parameters is dependent on variables that change with the patient and change over time.

Typically, if a patient will be performing the phototherapy at home, using a home-use device, that patient is given a prescribed therapy regime by his/her physician. The patient follows the strict time exposure regimen over a period of time, then schedules an appointment to meet with the supervising physician, who then adjusts the phototherapy regime based on parameters such as erythema, pain, redness, and the like. This process is imprecise for several reasons, including variable amounts of time between physician visits, and passage of time between the end of one therapeutic regime and review and adjustment of the regime by the physician. Also, there is often a significant burden for the patient to physically appear in a physician's office, or a clinic, to receive treatment. Such issues would be reduced using a phototherapeutic device adapted for home-use.

In addition, in home-use phototherapy devices, as with most devices that rely upon a light source to deliver a precisely controlled duration, intensity, and wavelength of light, the devices need these parameters to be calibrated to ensure desired light delivery. Calibrating the light source typically involves returning the unit to the manufacturer, or other service location, for remote calibration. While the device is being calibrated, the user cannot be using the device for treatment.

Thus, there remains a need for a convenient, reliable\system for enabling calibration of certain parameters of a physician office-use/home-use, phototherapy device. There also remains a need for a phototherapy device that includes a system for dynamically adjusting the therapeutic exposure parameters, based on real-time information provided by the patient using the device, and particularly for such a device permitting home-use, without direct presence of a physician or other medical personnel.

### SUMMARY

The present invention relates to phototherapeutic systems with an integral, or adapted for use with an, optical calibration system. One embodiment of the invention incorporates a controller within a housing and a hand-held light applicator, interconnected by a cable, and a calibration system built in to the housing of the device. The calibration system includes an off-axis optical sensor with a light-integrating cup that is used to measure the output of fiber-optic light, such as ultraviolet (UV) light, along a folded optical path. The folded optical path enables a relatively compact housing to be used, yet still achieve a relatively long path. Measuring light output from a multi-fiber light applicator at close proximity would require precise alignment of the fibers to the sensor and would tend to have a “hot spot” associated with each fiber. The integrating cup and off-axis optical sensor minimizes difficulties with alignment between each individual fiber of the applicator device and the optical sensor.

More specifically, in a preferred form, the present invention is a phototherapeutic system comprising a base unit, a phototherapeutic light assembly, and a power/control coupling assembly that couples the power supply and system controller to the light source controller. The base unit includes a base housing enclosing a power supply and system controller. The base housing includes an elongated recess extending along a calibration axis (C) to a sensor end and is configured to selectively receive and support the distal end of the applicator housing in a calibration position with the applicator axis substantially parallel to the calibration axis and with the distal end of the applicator housing opposite the sensor end of the recess. The base housing further includes: (1) a sensor for generating a detector signal representative of light incident thereon; and (2) a calibration assembly including a processor and a concave reflector, wherein the concave reflector is disposed at the sensor end of the recess and is adapted to collect light propagating along the calibration axis and incident thereon, and reflect at least a portion of the collected light to the sensor.

The phototherapeutic light assembly includes an elongated applicator housing extending along an applicator axis (A), and a light source and associate light source controller disposed within the applicator housing and adapted to generate and transmit light from a distal end of the applicator housing along the applicator axis in response to a control signal. The system controller is selectively operative to detect when the applicator housing is in the calibration position, and generate a control signal to selectively control the light source to generate light and to determine from the detector signal, a predetermined characteristic of the generated light.

In an embodiment of the present invention, the base housing includes a calibration slot adapted to removably receive the calibration assembly. In an embodiment, the processor generates the detector signal whereby the detector signal is representative of the spatial integral of light from the applicator and incident on the concave reflector.

In yet another embodiment of the present invention, the light source controller is a shutter blade activated by an electromagnetic solenoid to selectively, as desired, or for safety, interrupting light from the light source, to prevent emission from the applicator device.

The present invention further relates to a dynamic programmable phototherapeutic treatment system, wherein the treatment device is responsive to a stored treatment plan to generate a predetermined therapeutic UV light signal for application to a treatment surface region of a patient/user. The system comprises at least one patient/user input device responsive to information generated by a patient/user indicative of the patient/user's conclusion as to an effect of the UV light signal applied to the treatment surface region. The inventive system further comprises a patient/user controlled treatment plan adjuster responsive to the patient/user-generated information to modify one or more aspects of the stored treatment plan in a predetermined manner, preferably within allowed ranges.

In an embodiment of the inventive system, the patient/user's conclusion is one from a predetermined set of allowed conclusions. In another embodiment of the inventive system, this set of allowed conclusions includes a ranked hierarchy of sub-conclusions.

In another embodiment of the inventive system, the system further comprises at least one physician input device responsive to information generated by a physician indicative of a desired change to the stored treatment plan, and a physician-controlled treatment plan adjuster responsive to the physician-generated information to selectively control the permitted ranges, and/or other parameters, for one or more aspects of the treatment plan.

In yet another embodiment of the inventive system, the physician input device is remotely coupled to the physician-controlled treatment plan adjuster. In another embodiment, the physician input device is coupled to the physician-controlled treatment plan adjuster by way of the Internet.

Various embodiments may include any of the above described features, alone or in any combination. These and other features will be more fully appreciated with reference to the following detailed description, which is to be read in conjunction with the attached drawings.

### BRIEF DESCRIPTION OF THE DRAWINGS

FIG. 1 is a perspective view of a phototherapeutic system, including the off-axis optical sensor, of the present invention.

FIG. 1A is a functional block diagram for the system 100.

FIG. 2A shows a sectional view of the phototherapeutic light assembly of the embodiment of FIG. 1 in position for operation in the calibration mode.

FIG. 2B is a perspective view of a handheld phototherapeutic light assembly of the present invention.

FIG. 2C is a cross-section view of the phototherapeutic light assembly of FIG. 2B.

FIG. 3 is a perspective view of a light assembly of the present invention.

FIGS. 3A and 3B illustrate an embodiment of a light source controller comprising a shutter blade activated by an electromagnetic solenoid.

FIG. 4 is a perspective view of an off-axis integrating light meter of the present invention.

FIG. 5 is a side section view of the off-axis integrating light meter of the present invention.

FIG. 6 is a top perspective illustration of a light collection cup of the present invention.

FIG. 7 is a block diagram of an embodiment of the dynamic phototherapeutic treatment system of the present invention.

FIG. 8 is a flow-chart of an embodiment of the dynamic phototherapeutic treatment system and method of the present invention.

FIG. 9 is a representation of an exemplary interview screen of an embodiment of the inventive treatment system.

### DETAILED DESCRIPTION

The present invention is directed to systems and methods for providing phototherapy treatment to patients (or users), on an outpatient or home basis. The systems and methods can also be utilized in a physician's office or a clinic. It is designed to be used in the same way as in conventional, physician's office phototherapy, allowing for clearance of symptoms after 20-30 treatments, or in an accelerated mode that can lead to clearance in as few as 10 treatments.

“UVB light” as used herein, refers to the most therapeutically useful ultraviolet light in the treatment of psoriasis, namely narrowband UVB (311-315 nm), within the broad 280-320 nm UVB spectral band. However, in various embodiments, energy in other spectral ranges may be used. “Rx PIN” is a “Prescription Personal Identification Number” that identifies a treatment regimen associated with a specific user. “Tx code” is a treatment code associated with a specified user, and contains the information for the treatment regimen. “Treatment Regimen” is a set of parameters that defines a treatment plan, and typically includes data representative of an initial dose (spectral range, intensity, duration, interval for next treatment) and variations of those parameters for subsequent treatments, and a number of treatments.

Psoriasis clears (in most people) after an amount of UVB light has been deposited on the skin. UVB light produces a photo-biological response. The first stage of this response is erythema (reddening of the skin due to increased blood circulation). The dose that produces a minimal response observable by the eye is called Minimum Erythema Dose (MED).

Erythema (sunburn) is what determines how much UVB light can be given for a treatment. In conventional phototherapy, patients are given the minimal erythema doses (the lowest milliJoule dose that produces the onset of mild sunburn) with each treatment. Treatment typically is repeated every other day until the skin clears of the disease.

The “effective energy” associated with a treatment is the light energy that actually reaches the dermal layers. The skin increases the tolerance to UVB light by becoming thicker (hyperplasia) over the course of treatments. Because thicker skin attenuates more light, a dose must be increased in each subsequent treatment of a treatment plan for an effective aggregate amount of energy to reach the dermis. “Clearing” (of symptoms) is considered to be an 85-95% improvement. While 100% may happen, it generally is not an ultimate goal, because it can set patients up for disappointment and unrealistic expectations. Traditionally, the Starting Dose for whole body phototherapy starts at 1 MED (or a fraction, e.g., 0.75 MED). Localized treatments, however, can start at multiple MED's.

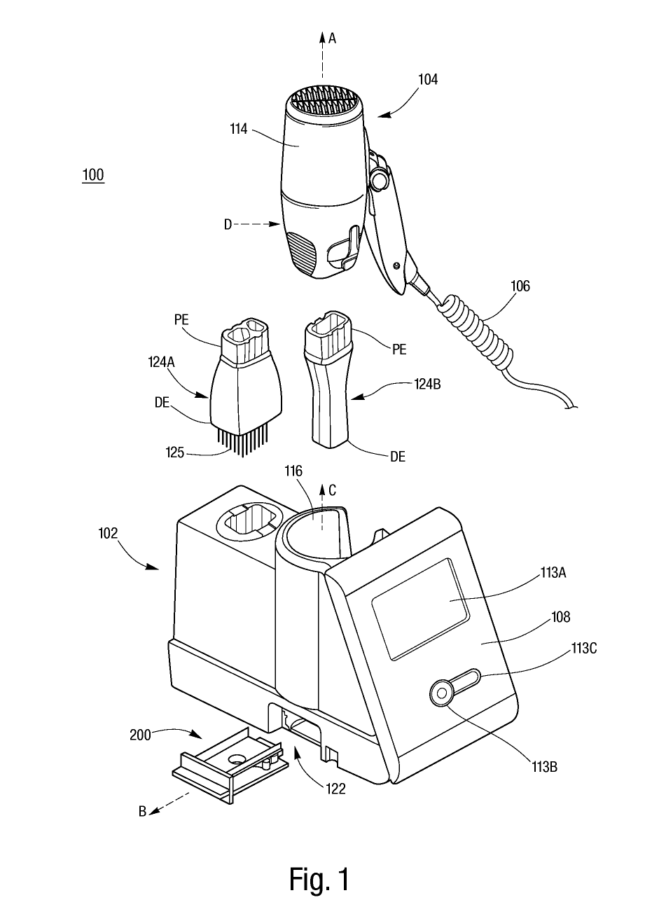

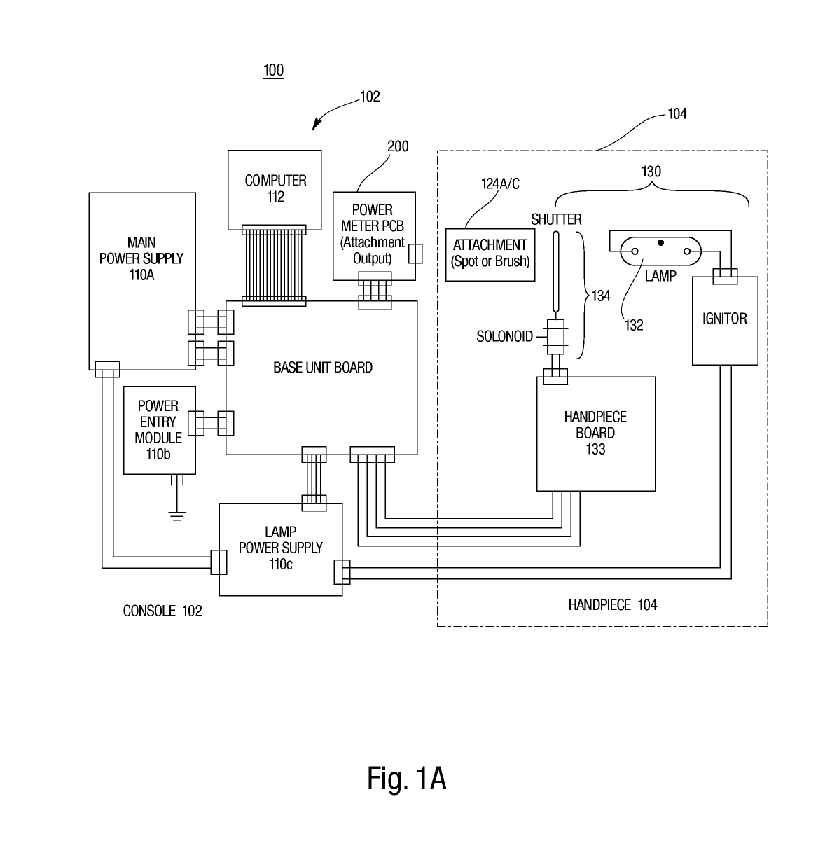

A phototherapeutic system that may be used in the present invention is shown in FIG. 1. In that illustrated embodiment, the phototherapeutic system 100 includes a base unit 102 and a phototherapeutic light assembly 104, and a power/control coupling assembly 106. FIG. 1A shows a functional block diagram for the system 100.

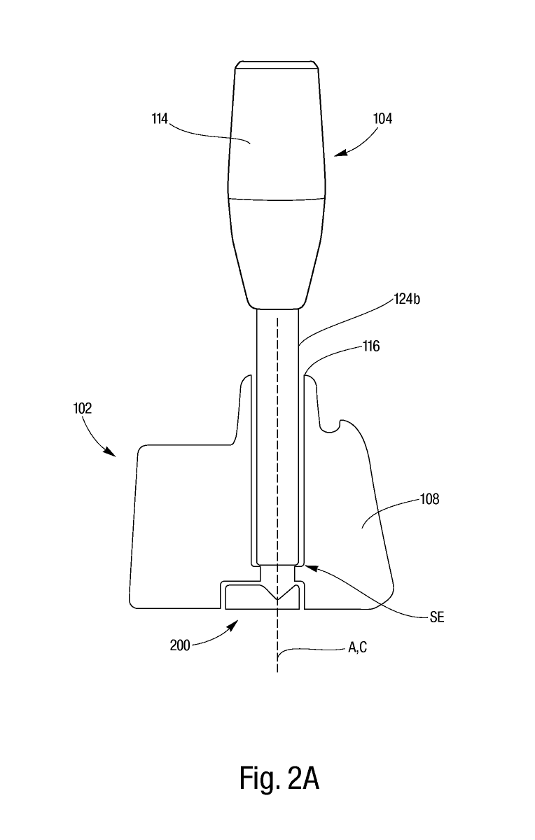

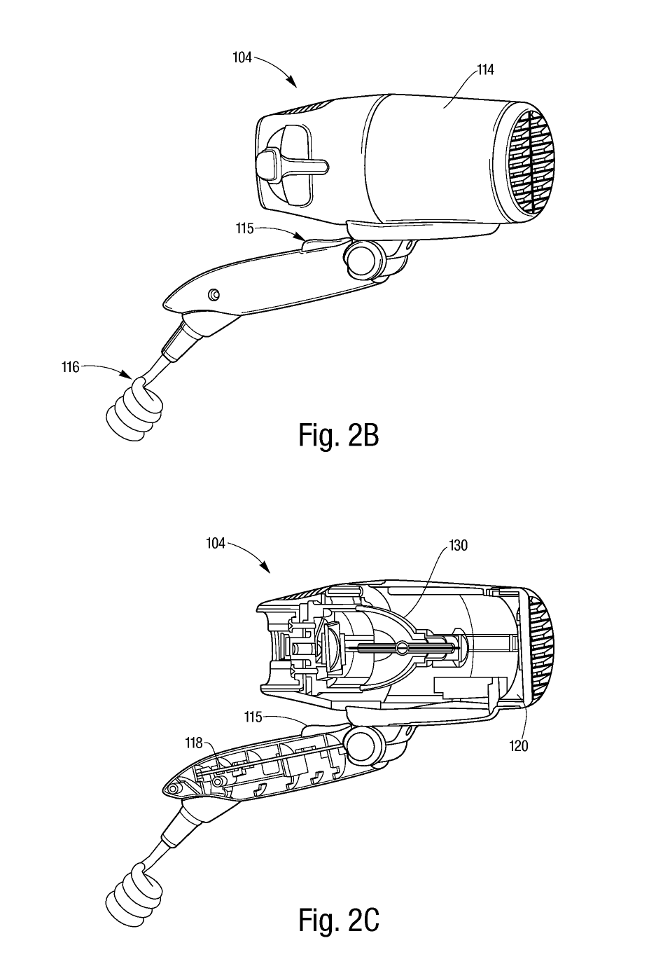

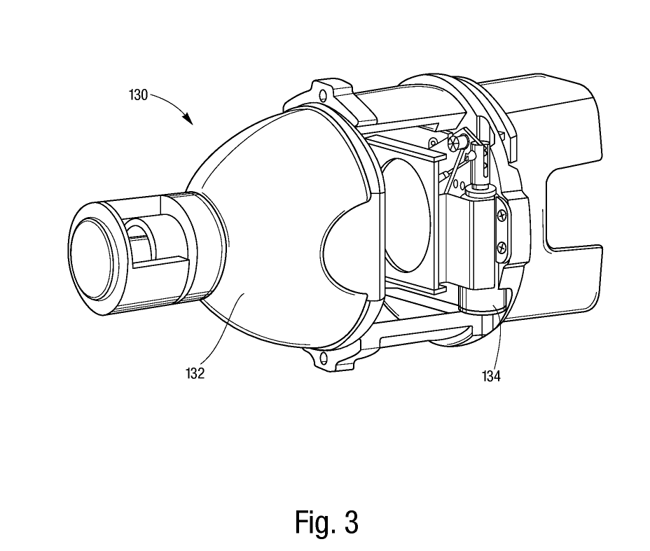

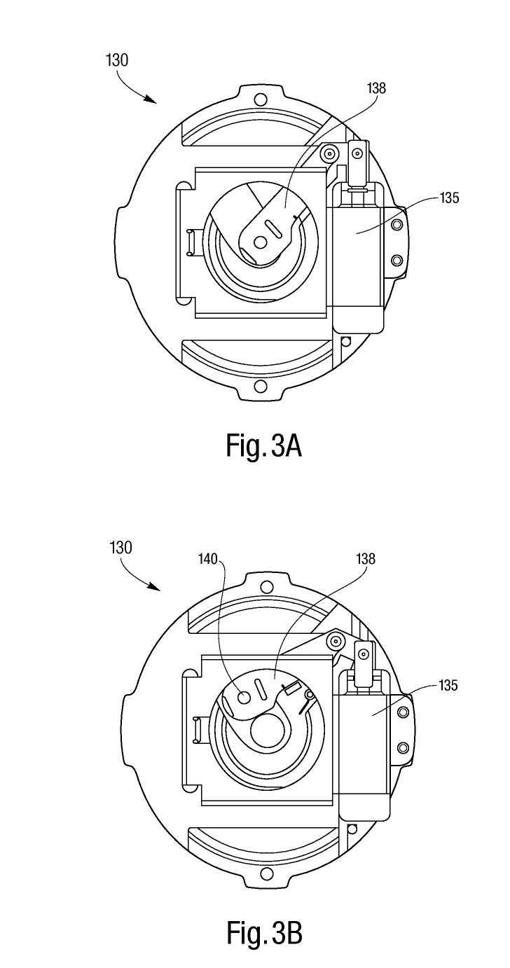

As shown in FIGS. 1 and 1A, the base unit 102 includes a base housing 108 enclosing a main power supply 110a, a power entry module 110b, and a lamp power supply110c. The power entry module 110b preferably is an AC input, the lamp power supply 110c provides power to the lamp assembly 130 (see FIG. 3), and the main power supply 110a provides power to all other components of the system. The base unit 102 further includes and a system controller 112, and including a user display, or GUI, 113A, which may be a touch-screen or other interactive display, a user-controlled input device 113B, and a slot 113C for insertion of a USB flash memory drive. The power supply 110 is a module that converts 110-220 volts line power to suitable DC power for supporting the various systems in device 100. The power supply 110 and the system controller 112 are connected by a power/control coupling assembly 106 which is coupled to light source controller 133 in phototherapeutic light assembly 104, as described below, and as illustrated in FIG. 2B.

The base unit 102 preferably is manufactured from rigid plastic, or other non-conductive material as commercially available, and may be pressure molded or assembled from individually manufactured elements.

As shown in FIG. 1, the illustrated phototherapeutic light assembly 104 preferably is ergonomically designed to optimize the shape and facilitate user use. The light assembly 104 includes an applicator housing 114, which extends along an applicator axis A to a distal end D. In the illustrated form of the invention, one of two light applicators 124a and 124b is adapted to these selectively/removably affixed to the distal end D of the housing 114. The light applicator 124a is commercially available under the trade name LiteBrush™, or may be constructed substantially as shown in FIG. 1, and is designed for the scalp and areas of the skin densely covered by hair. The light applicator 124b is commercially available under the trade name LiteSpot™, or may be constructed substantially as shown in FIG. 1, and is designed for relatively bare skin. Both of the illustrated light applicators 124a and 124b are elongated along an axis which, when a respective applicator is affixed to housing 114, is coaxial with axis A.

The applicators 124a and 124b are shaped to fit into a recess 116 in the base housing 108. The fiberoptic bristles of the LiteBrush™ handpiece 124a bypass hair that normally absorbs the light and blocks it from reaching the scalp. Uniform irradiation is achieved with smooth wavy combing action with the tips of the fibers in direct contact with the scalp. For skin not covered by hair, exposures are preferably tiled over the affected area with the LiteSpot™ handpiece 124b. The recess 116 extends along a calibration axis C to a sensor end SE, and is shaped to receive the applicator housing 114 with the distal end DE opposite the sensor and SE of the recess, as illustrated in FIG. 2A. The applicators 124a and 124b (and in some embodiments, the distal end of housing 114) and the recess 116 are elongated and a generally rectangular cross-section. However, other shapes may be used, such as circular, oval, substantially square, or any polygonal shape, so long as they are complimentary shapes so that the base unit 102 supports the housing in a calibration position, such as that shown in FIG. 2A. The applicator axis A of the phototherapeutic light assembly 104 substantially aligns with and is parallel with the calibration axis C of the recess 116, such that the distal end DE of the assembly applicator 124a or 124b extends through the recess and is opposite to a recess sensor end SE in a calibration position for performing system calibration.

The distal end D of the housing 114 of the light assembly is adapted to enable different phototherapeutic attachments 124a and 124b to be selectively and alternatively attached by a user for different phototherapeutic uses. Thus, the distal end D of housing 114 and the proximal ends PE of applicators 124a and 124b constitute a locking mechanism, such as a snap-on configuration to interchangeably connect with various attachments 124 as may be made available. It is important that the locking configurations create rigid, secure connections between the light assembly 104 and the various attachments 124 to ensure that, e.g., the optical fibers 125 of applicator 124a and the terminal aperture (not shown) of applicator 124b align with sufficient precision to ensure accurate calibration.

As shown in FIG. 2B, the light assembly 104 includes a trigger 115 for selectively activating the system, and the power/control coupling assembly 106. As shown in further detail in FIG. 2C, the light assembly 104 further includes lamp assembly 130, fan unit 120, and a handpiece control board 118.

The phototherapeutic light assembly 104 further includes a lamp assembly 130, as shown in the light assembly 104 of FIG. 2C, and as illustrated in further detail in FIG. 3. The light engine assembly 130, includes a light source 132, light source controller 133 (as shown in FIG. 1A), and associated light source output cut-off assembly 134. The light source 132 is controlled by the light source controller 133 in response to control signals from the system controller 112. In a calibration mode, a detector in housing 108 determines when the light assembly 104, with one of the light applicators 124a or 124b is positioned in the recess 116 as illustrated in FIG. 2A, and the light source 132 is selectively activated and calibrated, as discussed below.

In a preferred embodiment, the light source is a UV-B light source, for use in phototherapy treatment of certain diseases of the skin. Alternatively, the light source 132 may be one of several other therapeutic lamps, such as short arc lamp, metal halide lamp, xenon arc lamp, mercury xenon arc lamp, and light emitting diode, among others. The therapeutic wavelength may be achieved using a metal halide arc lamp, having a reflective surface, that directs light along the applicator axis A, and optionally through one or more optical filters (not shown).

As a safety feature, light from the light source 132 may be selectively interrupted, before being admitted from the light assembly 104. For example, during detection of improper action by the device, it may become necessary to shut all light from the light assembly 104 by way of light source cutoff assembly 134. In the illustrated embodiment, the light source cutoff assembly 134 includes a shutter 138 with an associated linkage connected to a solenoid plunger actuator. The solenoid plunger, with its associated linkage selectively moves a shutter 138 to enable and block light from the light source 132. FIG. 3A illustrates the shutter 138 impeding the light path from the light source. FIG. 3B illustrates the shutter 138 moved aside to no longer impede the light path to enable phototherapeutic treatment, or calibration. Except during calibration or active therapy, the shutter is closed, as shown in FIG. 3A, to block all UV output. The shutter element 138 may block all light entirely or may include an small pilot light opening 140 having an optical filter that allows only visible light to pass. In a preferred embodiment, the filter passes light above 395 nm wavelength. The light that passes through the pilot light opening 140 may be used to direct treatment, by enabling the user to visualize the position of the output pattern over the treatment area before allowing therapeutic light to pass.

The phototherapeutic system 100 further includes a calibration assembly 200 that is used in calibrating the system. Calibration is an important aspect of phototherapeutic systems, because total light output typically declines over time due to the intrinsic properties of the lamp and light pathways (e.g., optical fibers). Without frequent calibration, the treatment dose declines over time as the total light output declines. In the present invention, UVB output is measured before each treatment, and treatment exposure time is automatically adjusted by the system to achieve a target treatment dose. Typically, UVB output is measured in mW/cm² and dose is measured in mJ/ cm², where dose =UVB output x time (seconds). Thus, a decrease in power output results in an increase in exposure time (i.e., time that the shutter is open) during treatment.

page 10 of 26 LNLL-0116 FINAL 2-25-2011

As shown in FIG. 1, the calibration assembly 200 preferably is adapted to be selectively removed from the base unit 102 as may be required for maintenance or replacement. The calibration assembly 200 is designed to be of a shape to fit within a calibration assembly slot 122 in one side of the base housing 108, oriented along an axis B that is substantially perpendicular to the calibration axis C, such that the light emitted from the light assembly 104 along the calibration axis C hits a surface of the calibration assembly 200.

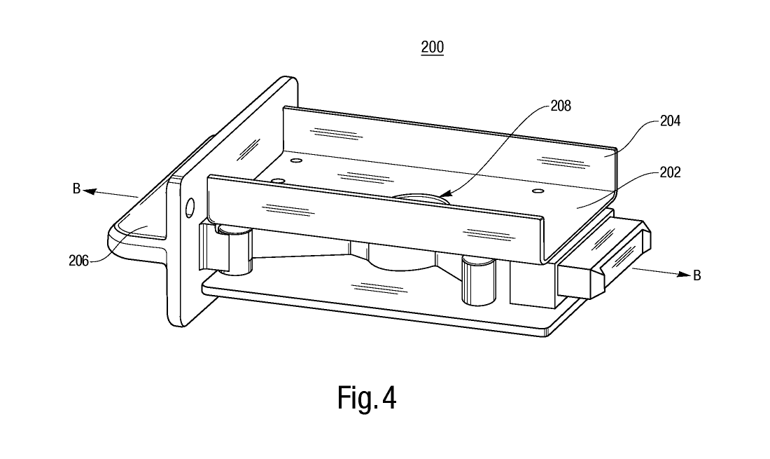

As shown in more detail in FIG. 4, the calibration assembly 200 includes a substantially support surface 202 that extends along the calibration assembly axis B. In the illustrated embodiment, the surface 202 includes perpendicular flanges 204 designed to assist the assembly 200 to slidably engage within the calibration assembly slot 122, to secure and precisely position the assembly 200 within the housing 108 during operation. Other methods and configurations of the assembly unit may be used, so long as it secures the surface 202 along an axis perpendicular to the calibration surface C.

The illustrated calibration assembly 200 further includes an assembly handle 206 that allows a user to easily slide the assembly 200 into and out of the calibrating slot 122. However, the assembly 200 may be manufactured with a variety of other handles, knobs, spring-releases, and release assemblies that permit a user to selectively remove the assembly 200 from the slot 122. In yet another embodiment, the assembly 200 is fixedly attached to the base unit 102. Although having the assembly 200 fixedly attached does not permit a user to remove the assembly 200 for maintenance and repair, it does not otherwise affect the functioning of the calibration assembly 200 as described herein.

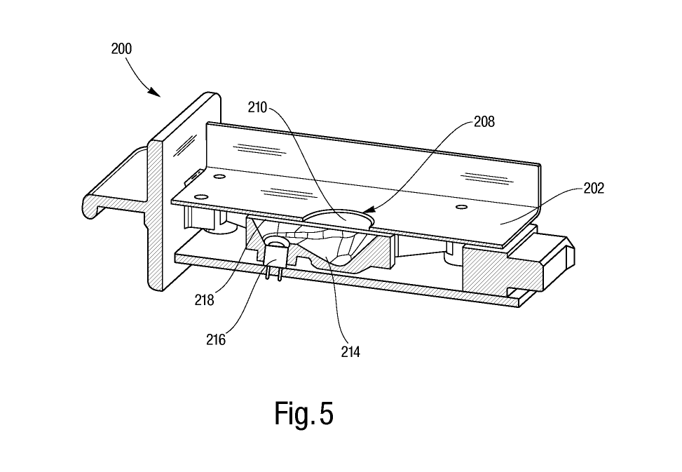

As shown in more detail in FIG. 5, the assembly surface 202 includes an aperture 208 of sufficient width to allow light from the light source 132 to enter from the distal end DE of the light applicator 124a (or 124b), along the calibration axis C through the aperture 208. In an embodiment of the invention, a diffuser 210 is positioned across the aperture 208 to diffuse light coming in from the light source 132. The diffuser enables the incoming light to disperse in a manner that allows more accurate reading during calibration. Other embodiments may omit the filter, depending on the nature of the light source and the sensitivity of the receiving light sensor.

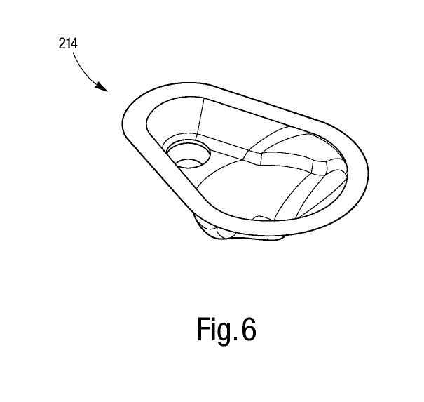

In an embodiment, the aperture 208 overlies a recess, which recess supports a structure having a concave surface in the form of a light integrating cup 214, substantially as shown in FIG. 4 and FIG. 5. In a preferred embodiment, as shown in FIG. 6, the integrating cup 214 is a concave collector having an optically reflective surface shaped so that light coming in through the aperture 208 is directed to a remote sensor device 216.

The illustrated embodiment of FIG. 5 includes a secondary reflector 218 positioned on the underside of surface 202, which in turn is opposite the sensor 216. This configuration established a folded optical path for the light incident on the concave reflective surface of integrating cup 216. That path extends from the reflective surface to the secondary reflector 218 and then to the detector 216. The concave reflective surface operates to integrate the collected light and focus by way of the secondary reflector 218 on to the sensor 216 In alternative embodiments, the secondary reflector 218 is absent, and the sensor 216 is positioned on the underside of surface 202 such that it receives the light collected and reflected by the integrating cup 214. In other embodiments, various geometries may be used, including hemispherical, pyramidal, and the like, that allow reflection of light to a relatively small sensor. Various positions of the sensor 218 may be used, depending on the geometry of the integrating cup, the nature of the light source, and the sensitivity of the sensor.

In an embodiment, the calibration assembly 200 or the sensor 216 apart from the assembly 200 is removed for calibration at a remote location.

In operation, in the calibration mode, a user positions the phototherapeutic light assembly 104 into the housing recess 116 such that the distal end DE of the light applicator 124a (or 124b) is in calibration alignment with the calibration assembly 200. The system controller 112 turns on power to the light assembly, and light then is directed through the light assembly 104, through the calibration assembly aperture 208, where the light passes through the diffuser 208 (if present) and onto the reflective surface of the concave light integrating cup 214, as shown in FIG. 6. At the light integrating cup 214, the light is collected and then reflected to either a secondary reflector 218, which directs the light to an underlying sensor 216, or directly to a sensor 216. In response to a signal from the sensor 216, the light source controller 133 can adjust the light from light source 132, or if out of permissible range, activate the solenoid to control shutter 138 to close for user safety.

By thus positioning the sensor 216 off-axis from the calibration axis C, it allows for measured power output to be independent of the light source alignment, instead integrating light from all of the fibers, eliminating effects from hot spots. The integration cup 214 essentially adjusts for any such misalignment by collecting, integrating, and directing the light to the sensor 216. In addition, the off-axis configuration of the calibration assembly 200, with its folded optical axis, permits the height of the housing 108 to be relatively small. In addition, the removable, modular calibration assembly 200 allows the calibration assembly to be relatively small and easily replaceable, making it more suited for use in relatively small, counter-top home-use systems. The specific positioning of the calibration assembly 200 becomes less important, provided that light coming in along the calibration axis C is directed into the concave reflected surface of the light integrating cup 214, which then can direct it to a sensor 216. In alternative embodiments, after reaching the integrating cup, the light may be directed one or more secondary reflectors before it reaches the sensor.

The sensor includes an associated microprocessor (not shown), which can be a part of the system controller 112, or the light source controller 133, or could be part of the calibration assembly 200, that processes and analyzes the incoming light to generate a detector signal representative of a predetermined characteristic of the generated light. In an embodiment, the microprocessor generates the detector signal of the sensor, which detector signal is representative of the spatial integral of light incident on the concave reflector.

In using one embodiment of the inventive system, each treatment regimen is identified by a Prescription PIN (Rx PIN) and a Treatment Code (Tx code). The Rx PIN acts as a treatment identifier and expires when the authorized number of treatments has been completed. The Tx code contains information for dosing, session frequency and a number of “authorized” sessions. The Rx PIN and Tx code are given to the patient by the prescribing physician in a conventional way, i.e. paper means, or stored in a USB flash drive. In an alternative embodiment, the user can enter the Rx PIN and Tx code via the touchscreen 113A on the base unit 102, as shown in FIG. 1. A sample of the RxPIN and Tx GUI is shown in Sample 1:

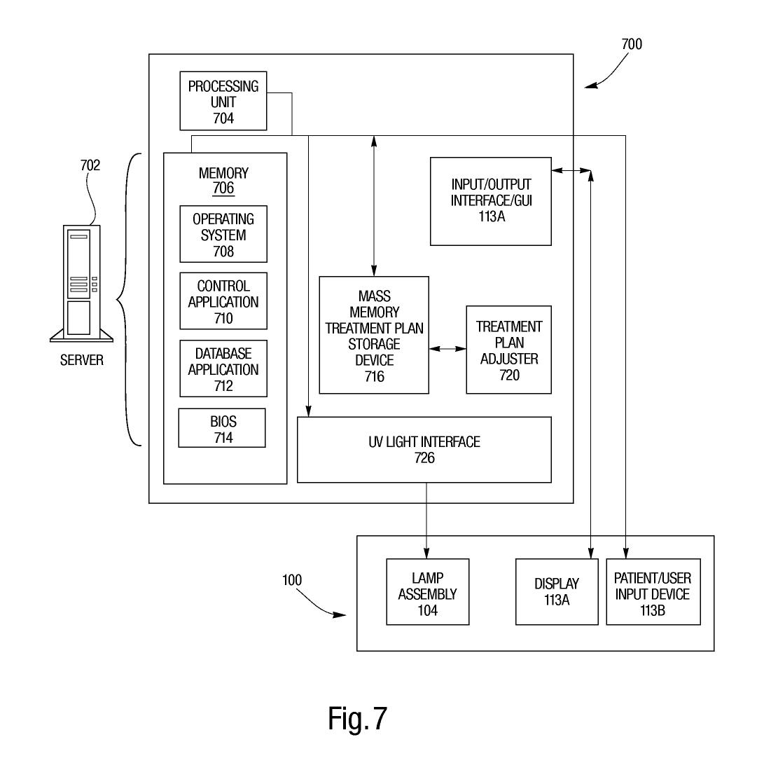

An exemplary embodiment of the treatment system of the present invention 700 is shown in FIG. 7, in block diagram form. The system includes a server 702, which includes at least a processing unit 704 and memory 706. Memory 706 typically includes an operating system 708, a control application 710, a database or other mass memory storage application, if required, 712, and a BIOS 714. In the present invention, the system 700 further includes mass memory treatment plan storage device 716 for storing a predetermined treatment plan, an input/output interface 718 that generates a graphical user interface (GUI) to the display device 113A certain treatment plan parameters, and a treatment plan adjuster 720. The system further includes a patient/user input device 722 to allow user input in response to output displayed on the display device 113A, and a UV light interface 226 for interfacing with the UV light source of the hardware system 100 and the lamp assembly 728. Each of the lamp assembly 104, display device 113A, and patient/user input device 113B are part of the hardware system 100.

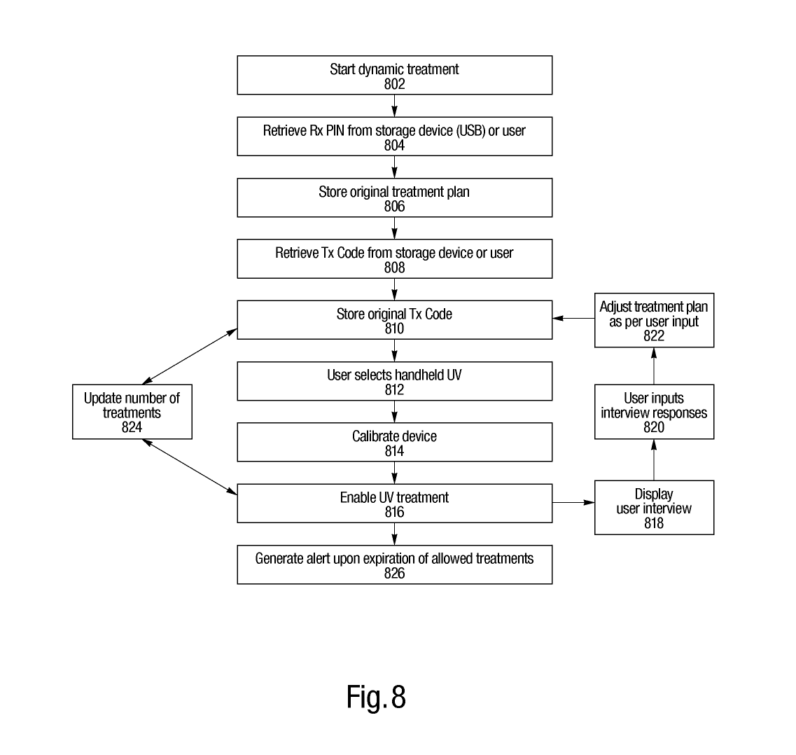

In the illustrated embodiment of the inventive system, and as shown further in the flowchart of FIG. 8, the treatment plan is started 802 by a user initiating the system—typically, by turning on the power to the base unit console 102. The RX PIN is retrieved 804, either from a storage device, such as a USB flash memory, or otherwise electronically, or manually input by the user. An original treatment plan is retrieved (from either the flash memory or, if connected by the Internet, from a physician or clinic) and is stored 806 in the mass memory device 716 in the form of a database, look-up table, or other formats as appropriate. The system then retrieves 808 a Tx Code (from, for example the flash memory), which includes user-specific parameters for the treatment plan, as described in further detail below. This Tx Code also is stored 810 in the mass memory device 716, or may be stored in a separate memory device. The specific order of retrieving and storing the Rx PIN and retrieving and storing the Tx Code may be performed in a different order without significantly affecting the performance of the inventive system.

Once the treatment information is retrieved and stored, the user selects 812 the attachment 124a or 124b, for handheld light assembly 104 which he/she wants to use for self-administering the UV treatment. This may also be performed by a physician or by an individual other than the actual patient. The system then calibrates 814 to ensure that the correct UV duration and intensity, and/or other parameters, are to be used for the treatment, for example, using the techniques described above.

Calibration 814 is performed by releasing the beam for a few seconds while the attachments 124a or 124b are positioned over a detector 216. The detector measures the light output and the control application 710 retrieves and stores this information in memory 716.

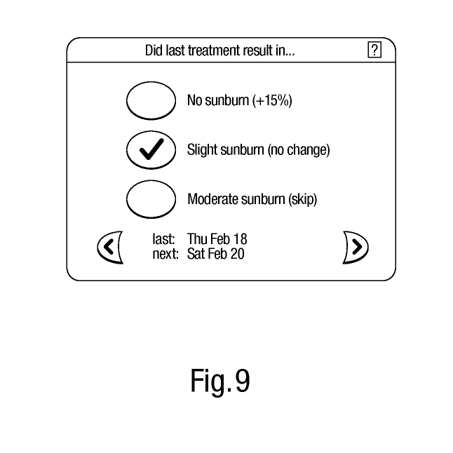

Once calibration 814 is complete, the UV light is activated and the UV treatment is enabled 816, and the patient/user aims the emitted light to the desired treatment region. After the first treatment, a user interview menu is displayed 818 to inquire of the user the effects of the treatment. An example of an interview display that may be used in the present invention is shown in FIG. 9. Such a display may include several fields, and make a range of inquiries into the effects of the phototherapy treatment. In the illustrated display, the user is asked to rate whether they experienced no sunburn, slight sunburn, or moderate sunburn. Responsive to the inquiries, the user inputs 820 his/her response to the interview screen. This input 820 may be via a touchscreen on the base unit console 102, via a mouse or other remote device, or other input systems and devices as may be generally commercially available.

Responsive to the user input 820, the inventive system adjusts the treatment plan 822. As shown in the illustrated example of FIG. 9, the treatment levels are adjusted upward by 15% if the user reports no sunburn, there is no change to the treatment plan if the user reports slight sunburn, and the next phototherapy session is skipped if the user reports moderate sunburn. The specific modifications to the treatment may vary depending on the nature of the original treatment plan, physician parameters, and other variables and factors unique to the type of therapy with which the inventive system is being used. A family member or friend may assist in the treatment for hard to reach areas of the body. As with any UV phototherapy treatment, clear UV blocking eyewear must be used by every person in the room during treatment.

In conjunction with the dynamic adjustment to the treatment plan as anticipated by the present inventive system, the number of treatments performed is tracked and updated 824 as the treatments are performed. The treatment plan typically includes a maximum number of treatments to be permitted, and once all of the treatments have been performed, the system generates an alert 826 notifying the user that no more treatments are available under that present plan. The user then returns to the physician for an updated or new Rx PIN and/ or TX Code to begin a new treatment cycle. The operation of the treatment system may, or may not, be disabled (from dispensing further phototherapy treatments) upon attainment of the “no more treatments are available under the present plan” condition.

In an embodiment of the system of the present invention, the time elapsed between treatments may also be monitored and stored, and the user then receives prompts that the treatment dose will decrease if the elapsed time warrants so.

In an alternative embodiment, the user may set the treatment dose each time, in some cases overriding does is set by the treatment plan. This mode is for expert users and is activated with a proper code. The prescribing doctor can decide whether the patient can safely use the system and allow activation. In this mode the dose is set manually, for example in milliJoules/cm², and can be adjusted at any time. In an embodiment of the system, the system may be configured to automatically set the exposure time needed for the desired dose.

Accordingly, there are at least two modes in which the system may be used: one is a prescription-based, predetermined treatment plan mode; the other is an advanced, user-determined mode. The differences between prescription and advanced modes are shown below in Table 1.

TABLE 1
Dose Setting Modes and Options

| | | | | | | Max. | |
|---|---|---|---|---|---|---|---|
| | | | | | Treat. | Number of | |
| | Prescription/ | Skin | Starting | Dose | Session | Treatment | Max. |
| Mode | Activation | Type | Dose | Increase | Frequency | Sessions | Dose |
| Prescription | Rx PIN | I-VI | Set by | Dose set | Set by the | Set by the | Internal |
| Mode | Tx code | Set | Doctor | by Doctor | Doctor. | Doctor. | Limit |
| | | by | | adjusted | Alert if | Alert to | per |
| | | Doctor | | per patient | Early/ | Patient if | Skin |
| | | | | input | Late | Exceeded | Type |
| Advanced | Specific | I-VI | Dose | Dose Set/ | Not Set | Not Set | System |
| Mode | Rx PIN for | Not | Set by | Adjusted | | | Max |
| | Advanced | Set | User | by User | | | |
| | Mode | | Each | Each | | | |
| | Activation | | Time | Time | | | |

In a preferred embodiment, each treatment regimen, or treatment plan has a unique Rx PIN. While each Rx PIN is unique, in one embodiment of the present system, the prescribing physician can give the same Tx code or a new one, based on the patient needs.

In an embodiment of the present invention, a Tx code is used, in addition to the Rx PIN to set the regimen at the desired levels. This code is in the form of a 10-digit number that contains all the necessary programming information. The prescription treatment plan in the USB drive suggests a default Tx code for each skin type. An example is shown in Table 2.

TABLE 2
Rx PIN Details

| | Rx PIN | |
|---|---|---|
| Parameter | significant digits | Tx Check Sum Digit |
| Description/ | 5 digits, assigned | Calculated by prescription |
| | by the doctor | Program |
| Example | 12345 | X |
| Rx PIN | 12345X | X = specific digit |
| | | (created by the program for validation to |
| | | prevent using unauthorized random PINs) |

In an embodiment, the Tx code includes the skin type, the initial starting dose, dose adjustments for each subsequent treatment, the treatment frequency and the total number of treatments to achieve clearing. The number of sessions needed to clear psoriasis can vary from a few to 30 or more, depending on the individual case and the regimen chosen.

The predetermined prescription treatment plan allows the physician to create a specific regimen for each patient. After a skin type is selected, default values for that skin type will populate the parameters. These values can be modified by the program user. Physicians can prescribe a pre-defined regimen based on skin type and past patient history.

TABLE 3
Tx Code Details

| | | | | | Number of | |
|---|---|---|---|---|---|---|
| | | | | Treatment | Treatment | |
| | Skin | | | Frequency | Sessions | Tx Check Sum |
| Parameter | Type | Dose | Regimen | (days) | Authorized | Digit |
| Property/ | Skin | Typical | Dosage | 2 = every | Total | Calculated by |
| Range/ | type | 20 to 360 | increase | other day | number of | prescription |
| Typical/ | I to VI | mJ/cm² | in %, i.e. | 7 = once | sessions | program |
| Limits | | | 10 = 10%; | per week | authorized | |
| | | | 20 = 20% | | (1-30). | |
| Example | 4 | 075 | 15 | 2 | 24 | X |

| | | |
|---|---|---|
| Tx code: | 407515224X | X = specific digit |
| | | (created by the |
| | | program for |
| | | validation and to |
| | | prevent using |
| | | unauthorized |
| | | random numbers) |

Table 4, below, lists an example of pre-defined Tx codes for each skin type. These Tx codes use the typical UVB MED of unexposed skin for that skin type, allow a 15% adjustment of the dose between treatments, schedules treatments for every other day, and allow a total of 20 treatments. Other parameters may be used in different embodiments, depending on the user, nature of the treatment, and the like.

The last column of the table gives the Tx code for a treatment plan that is on an accelerated schedule, i.e., an Accelerated Regimen. This regimen starts at twice the MED value for each skin type. Because the treatment with the present system is localized, using the handheld devices, most patients will tolerate an increased dose for the benefit of faster clearing.

TABLE 4
Recommended Tx Codes for Conventional and Accelerated Treatments

| | Tx code | Tx code |
|---|---|---|
| Skin Type | for Conventional Treatment | for Accelerated Treatment |
| I | 1 045 15 2 20 4 | 1 090 15 2 20 4 |
| II | 2 075 15 2 20 2 | 2 150 15 2 20 2 |
| III | 3 090 15 2 20 8 | 3 180 15 2 20 0 |
| IV | 4 120 15 2 20 5 | 4 240 15 2 20 8 |
| V | 5 150 15 2 20 3 | 5 300 15 2 20 6 |
| VI | 6 240 15 2 20 2 | 6 480 15 2 20 8 |

In an embodiment, an Advanced Mode Rx PIN is unique for each device and can be given to the patient with physician's authorization. The prescribing physician is responsible to determine whether a patient can be permitted to use the device in the Advanced Mode.

In alternative embodiments of the system of the present invention, the system may include a camera, which may be mounted on the base unit console 102, a standalone unit, integral with the console, or otherwise as available and appropriate at the time. The camera then may be used to collect direct information from the user regarding the level of erythema experienced from the treatment plan. The video data may be collected and transmitted directly to the physician's office, or may be stored locally for later transmission. The image may be transmitted via the Internet, via satellite, or downloaded to a hardware storage device, such as the USB, for transfer to the treating physician.

In another embodiment, the images from, e.g., a camera mounted in the handpiece, may be processed using software loaded on the device, which software is designed to determine skin type or erythema level (redness) and automatically adjust treatment levels, such as in a closed-loop system. Such a closed-loop embodiment would reduce the required amount of patient feedback, and would be particularly useful when a patient is treating multiple skin areas under different stages of treatment. Images could also be used to automatically match the shape of the light output, for example with appropriate optics and/or light sources, with the geometry of the affected area. Such a system would be particularly useful for vitiligo.

In another embodiment, an ultrasonic echo technique may be used to measure psoriasis plaque thickness. This information then is provided to the present system, and used as a metric in determining an applicable treatment plan.

### Examples

The present invention may further be understood by reference to the following example.

Options:

Define the starting MED using a standard MED test;

Choose from the table below; or

Use the standard starting dose according to protocols used in your facility. (Caution is advised because different manufacturers provide different equipment calibrations)

TABLE 5
Typical UVB MED of Unexposed Skin Using UVB-Select ™

| Type | Typical UVB-Select ™ MED of unexposed skin |
|---|---|
| I | 45 mJ/cm² |
| II | 75 mJ/cm² |
| III | 90 mJ/cm² |
| IV | 120 mJ/cm² |
| V | 150 mJ/cm² |
| VI | 240 mJ/cm² |

Notes:

(i) The values of the table are averages for individuals with the same skin type and for a typically unexposed area, like the buttocks or abdomen. Chronically sun-exposed areas of the body will usually require higher MED's.

(ii) Due to spectral variations between fluorescent tube based phototherapy devices and the filtered, UVB-Select output of Levia, the MED of a specific patient with the present system will likely be different to the MED values measured with other devices.

(iii) Different companies provide meters with different calibrations. It is possible that one calibration is substantially different from the calibration of equipment from other companies. This why MED testing using the present system is recommended before starting treatment.

(iv) There are variations between individuals with the same skin color because skin thickness is a factor in UV light tolerance. In order to accurately control the dose and ensure patient safety and comfort, the MED should be determined for each patient before treatment using a standard MED test.

(v) MED test: Deposit doses around the typical MED for the skin type of the patient and observe the response 24 hours after test doses are delivered. Typically a patient is given 0.5, 0.75, 1.00, 1.25, 1.50, 2.00 times the typical MED for his/her skin type.

(vi) MED test using the inventive system: Use the unit in Advanced Mode and insert the LiteSpot attachment. Use Adjustment Dose to set the dose levels at the MED fractions and multiples given above. Expose the buttocks or stomach of the patient with each of these doses and use a marker to mark on the skin the dose of each spot. Evaluate the response 24-48 hours after the test. The minimum dose that produces visually detectable erythema is the MED for this patient.

Each treatment increases the skin tolerance to UVB light and a higher dose is needed each time to produce erythema. The recommended rate of adjustment is 15% per treatment for the standard three days/week clearing schedule.

The determining factor for the dose adjustment is the response to the previous treatment. Slight or no response require an increase, mild to marked response should leave the dose unchanged, moderate or severe response should recommend that the treatment be skipped until the skin recovers.

TABLE 6
Dose Adjustment Tables

| Erythema | | Pain | |
|---|---|---|---|
| Grade | Observation | Grade | Sensation/Pain Level |
| 0 | No erythema or pinkness | 0-3 | None to Perceptible |
| 1 | Slight pink-mild erythema | 4-5 | Slight Discomfort |
| 2 | Marked erythema, no edema | 6-7 | Moderate Discomfort |
| 3 | Fiery erythema with edema | 8-10 | Severe Discomfort |
| 4 | Fiery erythema, with | | and Pain |
| | edema/blisters | | |

| | Pain | | |
|---|---|---|---|
| Erythema | 0-3 | 4-7 | 8-10 |
| 0 | + | + | + |
| 1 | + | + | 0 |
| 2 | 0 | 0 | − |
| 3 | 0 | − | − |
| 4 | − | − | − |

If the Dose Adjustment of the Tx code has been set to a different value than 15%, the dose will be adjusted by the set percentage.

If a higher starting dose is chosen (above 1 MED) and kept constant between treatments, improvement may be observed faster. However if after a number of treatments the skin tolerance is such that the (constant) dose is not sufficient to produce erythema, treatment will not be effective.

In an embodiment of the present invention, the computer keeps track of the time between treatments. If treatments are skipped for more than a week, the skin begins to lose its photo-protection by sloughing off these cells. The inventive system may make adjustments based on the elapsed time, as shown in the following Table 7.

TABLE 7
Elapsed Time Adjustments

| Elapsed | Built-up | |
|---|---|---|
| Time | Protection | |
| (weeks) | Loss | Dose adjusted to: |
| 1 | 25% | Last dose minus |
| | | 25% of total increase |
| 2 | 50% | Last dose minus- |
| | | 50% of total increase |
| 3 | 75% | Last dose minus- |
| | | 75% of total increase |
| 4 | 100% | Starting dose |
| (or more) | | |

The following Table 8 is a table of recommended maximum doses for skin type that may be used in setting the treatment plan or making dynamic adjustments in accordance with the present invention:

TABLE 8
Maximum Dose Values Available

| Skin Type | Max. Dose |
|---|---|
| I | 400 mJ/cm² |
| II | 500 mJ/cm² |
| III | 600 mJ/cm² |
| IV | 700 mJ/cm² |
| V | 800 mJ/cm² |
| VI | 990 mJ/cm² |

Localized treatment dose and multiple MED doses: When treating the whole body with light panels or booths, it is important to keep the dose at the MED level because whole body sunburn will produce substantial discomfort and potentially other complications.

When treatment is applied to small, targeted areas, localized discomfort/pain is the limiting factor. If the patient can tolerate higher MED doses, the skin will clear faster. Local creams and topical anesthetics can provide relief in these situations. Since the skin will take longer to recover, the treatments can be spread to rates of up to once a week.

Treatments should never exceed 6 MED's (the localized MED of the skin with the built up tolerance) because such a dose may produce blisters of not just normal skin but also psoriatic plaques.

Preservation Treatment: The time interval during which the patient will remain clear depends on many factors. For preservation it is recommended that the patient continues treatment once a week at the ending dose. Additional home care (moisturizing, etc.) can also prolong remission.

Phototherapy should not be applied to the genital area except with the explicit order of the physician and should be monitored diligently. There is an increased risk of burning because the genital skin is thin.

It will occur to those skilled in the art, upon reading the foregoing description of the preferred embodiments of the invention, taken in conjunction with a study of the drawings, that certain modifications may be made to the invention without departing from the intent or scope of the invention. It is intended, therefore, that the invention be construed and limited only by the appended claims.

We claim:

1. A phototherapeutic system comprising:
A. a base unit including a base housing enclosing a power supply and a system controller;
B. a phototherapeutic light assembly including an elongated applicator housing extending along an applicator axis, and a light source and associated light source controller disposed within the applicator housing and adapted to generate and transmit light from a distal end of the applicator housing along the applicator axis in response to a control signal; and
C. a power/control coupling assembly coupling the power supply and system controller to the light source controller,
D. wherein the base housing includes an elongated recess extending along a calibration axis to a sensor end and is configured to selectively receive and support the distal end of the applicator housing in a calibration position with the applicator axis substantially parallel to the calibration axis and with the distal end of the applicator housing opposite the sensor end of the recess,
E. wherein the base housing further includes: (i) a sensor for generating a detector signal representative of light incident thereon; and (ii) a calibration assembly including a processor and a concave reflector, wherein the concave reflector is disposed at the sensor end of the recess and is adapted to collect light propagating along the calibration axis and incident thereon, and reflect at least a portion of the collected light to the sensor, and
F. wherein the system controller is selectively operative to detect when the applicator housing is in the calibration position, and generate a control signal to selectively control the light source to generate light and to determine from the detector signal, a predetermined characteristic of the generated light.

2. A phototherapeutic system according to claim 1, wherein the base housing includes a calibration slot adapted to removably receive the calibration assembly.

3. A phototherapeutic system according to claim 1, wherein the processor generates the detector signal whereby detector signal is representative of the spatial integral of light incident on the concave reflector.

4. A phototherapeutic system according to claim 1, wherein the light source controller is a shutter blade activated by an electromagnetic solenoid.

5. A phototherapeutic system according to claim 1, wherein the calibration assembly is removably attached to the base housing for calibration of the calibration assembly at a location remote from the base housing.

6. A dynamic programmable phototherapeutic treatment system, wherein the treatment device is responsive to a stored treatment plan to generate a predetermined therapeutic UV light signal for application to a treatment surface region of a patient/user, comprising:
A. at least one patient/user input device responsive to information generated by a patient/user indicative of the patient/user's conclusion as to an effect of the UV light signal applied to the treatment surface region; and
B. a patient/user controlled treatment plan adjuster responsive to the patient/user-generated information to modify one or more aspects of the stored treatment plan in a predetermined manner within allowed ranges.

7. A phototherapeutic treatment system according to claim 6, wherein the patient/user's conclusion is one from a pre-determined set of allowed conclusions.

8. A phototherapeutic treatment system according to claim 7, wherein the set of allowed conclusions includes “skin redness” and “pain”.

9. A phototherapeutic treatment system according to claim 8, where in at least one of the allowed conclusions includes a ranked hierarchy of sub conclusions.

10. A phototherapeutic treatment system according to claim 6, further comprising:
A. at least one physician input device responsive to information generated by a physician indicative of a desired change to the stored treatment plan; and
B. a physician controlled treatment plan adjuster responsive to the physician-generated information to selectively control the permitted ranges for one or more aspects of the treatment plan.

11. A phototherapeutic treatment system according to claim 10, wherein the physician input device is remotely coupled to the physician-controlled treatment plan adjuster.

12. A phototherapeutic treatment system according to claim 11, wherein the physician input device is coupled to the physician-controlled treatment plan adjuster by way of the Internet.

13. A phototherapeutic treatment system according to claim 6, wherein the treatment plan adjuster modifies the plan based on the elapsed time between treatments.

14. A phototherapeutic treatment system according to claim 6, wherein the treatment plan adjuster modifies the plan based on the elapsed time between treatments and to information generated by a patient/user.
